(19) United States
(12) Patent Application Publication
(10) Pub. No.: US 2013/0090683 A1
(43) Pub. Date: Apr. 11, 2013

(54) SYSTEM FOR MAINTAINING OR ALTERING THE BODY TEMPERATURE AND METHOD OF USE

(71) Applicant: LIFE RECOVERY SYSTEMS HD, LLC, Kinnelon, NJ (US)

(72) Inventor: Robert B. Schock, Sparta, NJ (US)

(73) Assignee: LIFE RECOVERY SYSTEMS HD, LLC, Kinnelon, NJ (US)

(21) Appl. No.: 13/645,935

(22) Filed: Oct. 5, 2012

Related U.S. Application Data
(60) Provisional application No. 61/543,499, filed on Oct. 5, 2011.

(51) Int. Cl. A61F 7/00; A61B 17/132

(52) U.S. Cl. CPC A61F 7/0053; A61B 17/132
USPC 606/203; 607/104

(57) ABSTRACT

A system for maintaining and altering the body temperature of a user includes a garment defining an interior space adapted to conform to and enclose a torso of the user. The garment includes at least one fluid passage configured to channel a heat transfer fluid into direct contact with the torso of the user. The system includes a portable pump for circulating a heat transfer liquid through the at least one fluid passage of the garment and a portable blower apparatus for circulating a heat transfer gas through the at least one fluid passage of the garment.

### CROSS REFERENCE TO RELATED APPLICATION

This application claims the benefit of U.S. Provisional Application No. 61/543,499 filed Oct. 5, 2011, which is incorporated herein by reference in its entirety.

### BACKGROUND

The field of the subject matter disclosed herein relates generally to systems for maintaining and/or altering the body temperature of a user and, more particularly, to systems and methods that enable maintaining the body temperature at a predetermined temperature of a user and/or the quick and efficient adjustment of the body temperature of the user, e.g., to induce hypothermia.

According to the Centers for Disease Control and Prevention, each year an estimated 1.4 million people in the United States sustain a Traumatic brain injury (TBI). Among those that sustain a TBI, approximately 50,000 die, 235,000 are hospitalized, and 1.1 million are treated and released from the hospital. In a domestic setting, a TBI is often the result of an automobile accident. This tragedy commonly strikes young individuals and results in tremendous levels of disability and financial loss. In modern military combat situations, a TBI is often the result following an attack where an improvised explosive device (IED) has been used. In previous combat settings, victims of explosive attacks often died due to penetrating injuries to the thorax and head, but modern body armor and protective helmets now allow many victims of explosive attacks to survive. Unfortunately, a significant number of survivors who have sustained a TBI develop long-term neurological disabilities. One of the current treatments to mitigate injury is to perform a craniotomy to relieve the pressure in the injured brain. Improved therapies to deal with TBIs are needed.

Prior research has shown that rapid cooling of the brain may help to prevent long-term injury. In an animal model, it was observed that if cooling was accomplished within 60 minutes of TBI, both edema and injury were significantly reduced. (Markgraf et al., “Treatment window for hypothermia in brain injury,” J Neurosurg. 1995(6):979-83 (Dec. 2001)). On the other hand, if cooling did not occur until 90 minutes after injury, it offered no significant benefit. Clinical studies in which patients with head injuries have been cooled have generally not been encouraging, but these have only used slow cooling methods that missed the 60-minute window for treatment by multiple hours. (Clifton, Guy L., “Is keeping cool still hot? An update on hypothermia in brain injury,” Current Opinion in Critical Care 10(2):116-9 (Apr. 2004)). These observations have led researchers to consider therapeutic hypothermia as a possible treatment for reducing the adverse consequences of TBIs. Various studies have shown that mild systemic hypothermia (cooling the body by approximately 3-5 degrees Celsius (° C.) (5.4-9.0 degrees Fahrenheit (° F.))) may reduce damage to vital organs, including the brain.

Rapid cooling of the brain in the field, particularly in a combat setting, is a challenge. Victims of IED attacks often have severe injuries to the extremities. These injuries are usually associated with life-threatening bleeding. Conventional systems used for rapid cooling cover most of the body and would be inappropriate in these situations; e.g., the coolant may introduce a risk of infection and may impede blood-clotting mechanisms. A preferred approach would be to cool the brain selectively, while preventing cooling of the extremities.

Cooling of the brain alone, however, is difficult to achieve. The primary challenge is that the warm blood from the core of the body (the heart, lungs, and other intra-thoracic organs) is constantly pumped to the interior of the brain. This limits the effectiveness of conventional cooling devices such as cooling caps and intra-nasal cooling devices, which cool the head only. There is a need for a portable device that can cool the entire body core to reduce brain temperature rapidly, which may produce a higher rate of patient salvage.

### BRIEF DESCRIPTION

In one aspect, a system for maintaining and altering the body temperature of a user generally comprises a garment defining an interior space adapted to conform to and enclose a torso of the user. The garment comprises at least one fluid passage configured to channel a heat transfer fluid into direct contact with the torso of the user. A portable pump is provided for circulating a heat transfer liquid through the at least one fluid passage of the garment, and a portable blower apparatus is provided for circulating a heat transfer gas through the at least one fluid passage of the garment.

In another aspect, a system for maintaining and altering the body temperature of a user generally comprises a garment defining an interior space adapted to conform to and enclose a torso of the user. The garment has a pair of arm openings and a pair of leg openings. The garment comprises at least one fluid passage configured to channel a heat transfer fluid into direct contact with the torso of the user. The system also includes a portable pump for circulating the heat transfer fluid through the at least one fluid passage of the garment. A tourniquet is positioned at each of the leg and arm openings and is selectively moveable between an inoperable and an operable position wherein the tourniquet is capable of reducing the blood flow to the respective arm or leg.

In yet another aspect, a system for maintaining and altering the body temperature of a user generally comprises an outer body armor garment configured to protect at least a torso of the user and a garment for placement under the outer body armor garment. The garment defines an interior space adapted to conform to and enclose the torso of the user. The garment comprises at least one fluid passage configured to channel a heat transfer fluid into direct contact with the torso of the user. A helmet is configured to protect the head of the user and comprises a liner having at least one fluid passage configured to channel the heat transfer fluid into direct contact with the head of the user. The system further includes a portable pump for circulating the heat transfer fluid through the at least one fluid passage of garment and the at least one fluid passage of the liner.

### BRIEF DESCRIPTION OF THE DRAWINGS

FIG. 1 is a schematic of an exemplary cooling system for adjusting the body temperature of a user.

FIG. 2 is a front view of an exemplary garment of the cooling system of FIG. 1.

FIG. 3 is a front view similar to FIG. 2 but illustrating the garment under conventional military body armor.

FIG. 4 is a cross-sectional view of a laminate structure used to form the garment of FIG. 2.

FIG. 5 is a schematic view of one embodiment of the body-facing layer of the garment having fluid passages formed therein.

FIG. 6 is a cross-sectional view of an exemplary built-in tourniquet for arm and leg openings of the garment.

FIG. 7 is a perspective view of another exemplary tourniquet suitable for use with the garment.

FIG. 8 is a schematic of a portable control unit of the cooling system of FIG. 1.

FIG. 9 is a schematic of one embodiment of the cooling system including an outlet pump configuration.

FIG. 10 is a schematic of another embodiment of the cooling system including an inlet pump configuration.

FIG. 11 is a schematic of a portable control unit integrated with a valve control system and sensors for distributing heat transfer fluid within the garment.

FIG. 12 is a schematic of another embodiment of the portable control unit integrated with a valve control system and sensors for distributing the heat transfer fluid within the garment.

FIG. 13 is a schematic of one embodiment of the exemplary cooling system including a head-cooling device.

FIG. 14 is a schematic of another embodiment of the cooling system including a head cooling device hood.

FIG. 15 is a schematic of a blower apparatus connected to the garment of FIG. 2.

Corresponding reference characters indicate corresponding parts throughout the drawings.

### DETAILED DESCRIPTION OF THE DRAWINGS

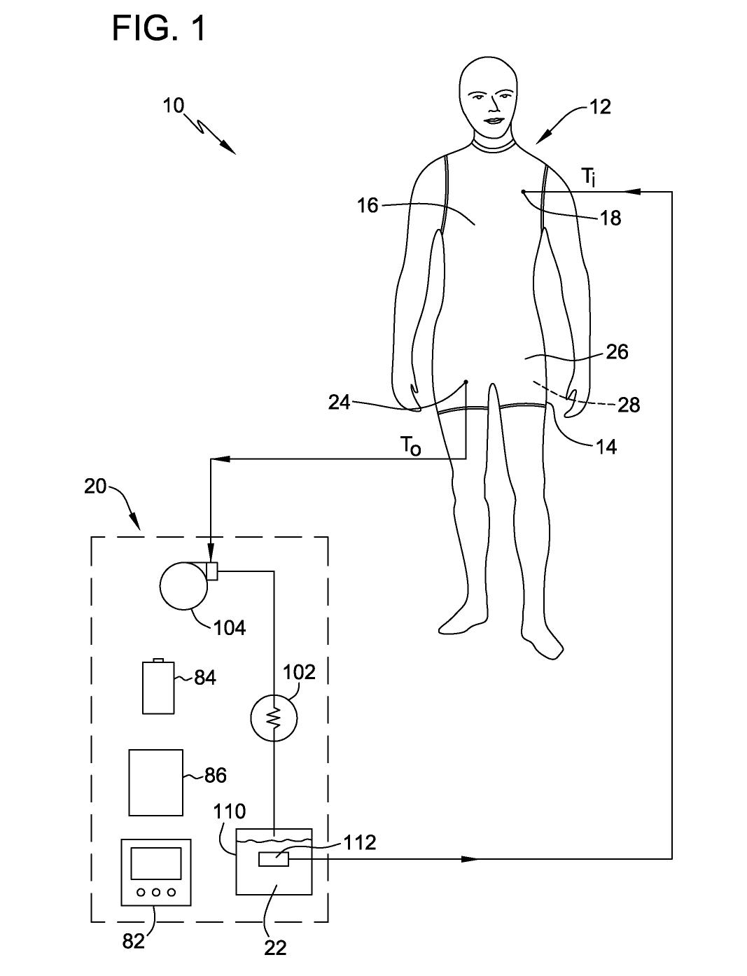

Referring now to the drawings, FIG. 1 illustrates an exemplary cooling system, indicated generally by 10, for adjusting and/or maintaining the body temperature of a user. The cooling system 10 generally includes a garment 12 defining an interior space 14 for receiving at least a portion 16 of the body of a user. The garment 12, seen in FIG. 1, e.g., is configured for substantially sealingly enclosing the body portion 16 of the user (illustrated in FIG. 1 as the torso of the user) within the interior space 14 with the garment generally contiguous with the body portion. More particularly, the garment 12 is adapted to generally conform to the body portion 16 disposed within the interior space 14. At least one inlet 18 of the garment 12 is configured to be selectively attachable to a portable control unit 20 to receive a heat transfer fluid 22 into the garment for direct fluid contact with the body portion 16. The heat transfer fluid 22 may include at least one of a liquid, such as water, saline, or other biocompatible liquid, and a gas, such as warmed or cooled air. The inlet 18 is connected in fluid communication with the interior space 14 of the garment 12 to channel the heat transfer fluid 22 into the interior space 14 for flowing over the body portion 16 in direct contact therewith to promote heat transfer between the body portion 16 and the heat transfer fluid 22. The garment 12 also has at least one outlet 24 in fluid communication with the interior space 14 of the garment for draining the heat transfer fluid 22 from the garment 12. In the illustrated embodiment, the inlet 18 and outlet 24 are positioned on the garment 12 such that upon enclosure of the body portion 16 within the interior space 14, the inlet 18 faces a side of the body portion 16 opposite the outlet 24.

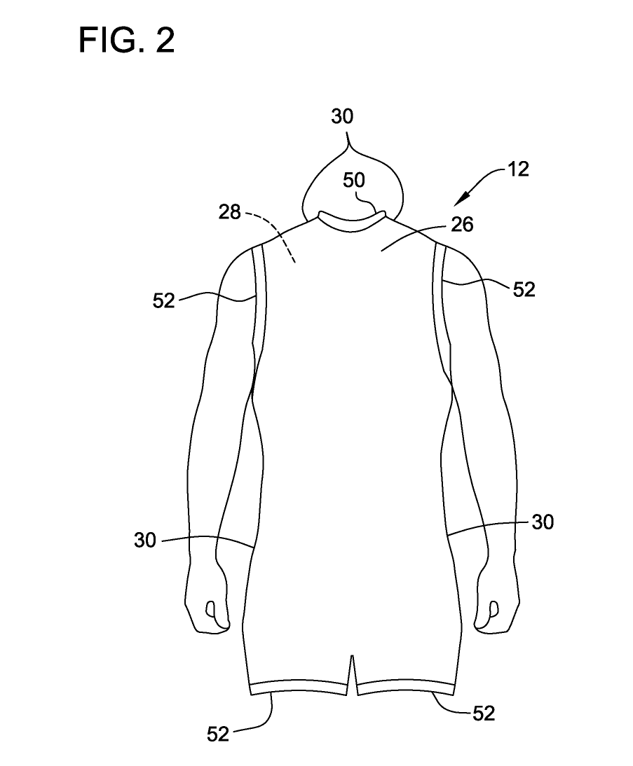

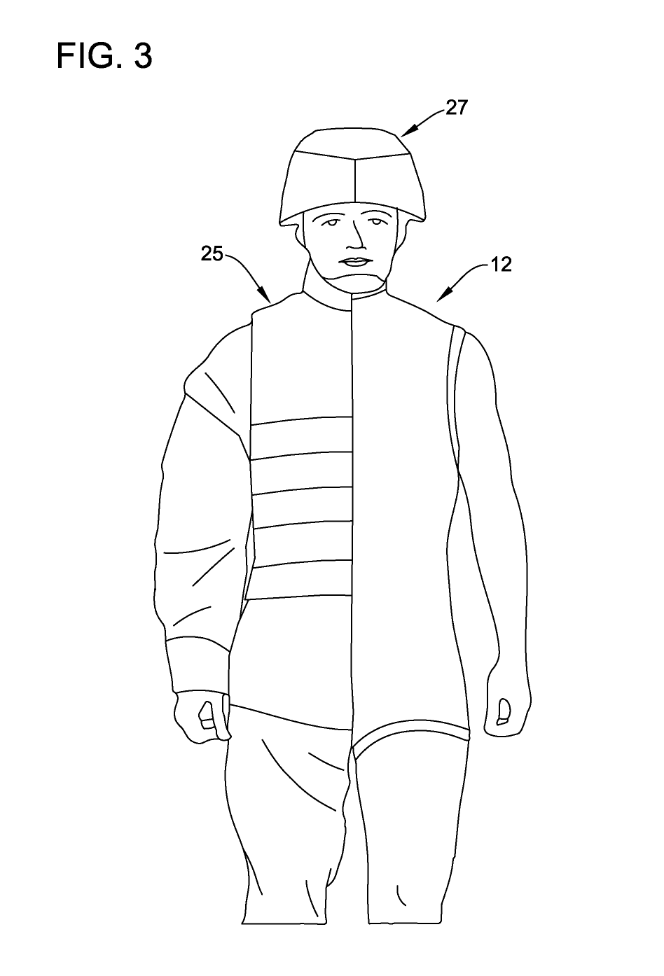

FIG. 2 is a front view of an exemplary garment 12 of the cooling system 10 of FIG. 1. While illustrated as a singlet-like undergarment, it is understood that the garment 12 is not limited to the shape, dimensions, or even garments illustrated herein. Instead, a garment as used herein is contemplated to include, but not be limited to, articles of clothing worn over/on a portion of the body. For example, a garment may include a shirt having long sleeves or short sleeves, pants, tights, full body suits, and the like. FIG. 3 is a front view similar to FIG. 2 but showing the garment 12 under conventional military body armor garment 25. As seen in FIG. 3, the user is wearing a conventional military helmet 27 in addition to the body armor garment 25.

In the embodiment seen in FIG. 2, the garment 12 includes at least a front panel 26 configured to cover at least a portion of the front of the torso of the user wearing the garment, and a back panel 28 configured to cover at least a portion of the back of the torso of the user wearing the garment. The front panel 26 and back panel 28 may be joined together at a plurality of continuous seams (not shown) along the outer edges 30 of the panels 26, 28 by, for example, sewing, adhesive bonding, heat welding, or other bonding technique along the length of the seam. The plurality of seams are configured to prevent fluid from passing through the seams.

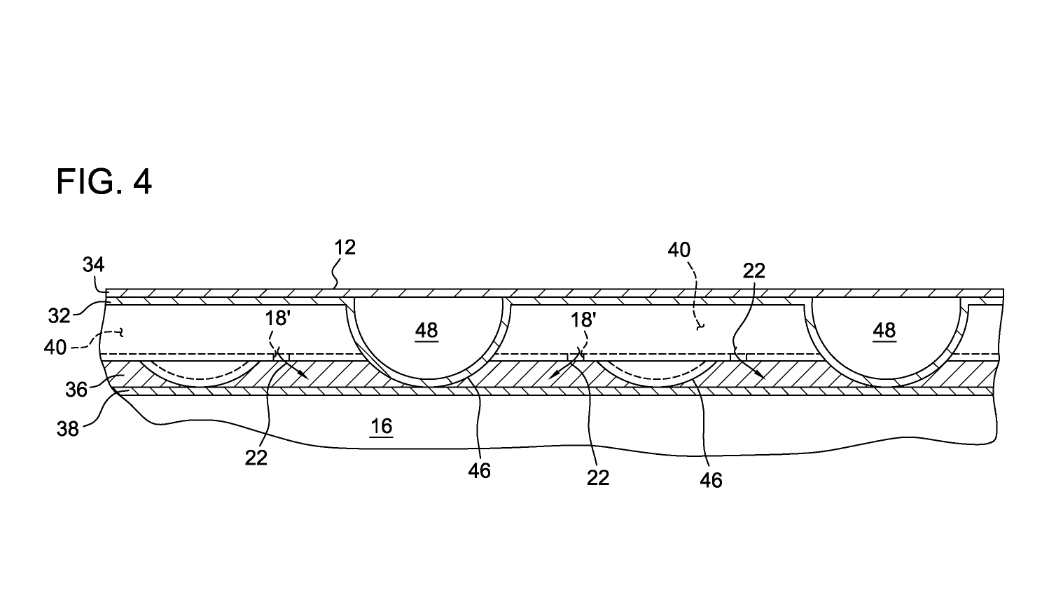

FIG. 4 is a cross-sectional view of a suitable laminate structure used to form the garment 12. In the illustrated embodiment, each of the front panel 26 and the back panel 28 are formed from the laminate structure. The illustrated laminate structure includes at least an inner body-facing layer 32 and a fluid impermeable outer layer 34 configured for face-to-face engagement with each other. The layers 32, 34 are joined to each other along their facing surfaces to form at least one fluid passage 40 between the layers 32, 34. The fluid passage 40 is configured for fluid communication with at least one inlet 18 for receiving the heat transfer fluid 22. The body-facing layer 32 has at least one opening 18′ (or sub-inlet) corresponding to the fluid passage 40 for allowing the heat transfer fluid 22 to pass from the fluid passage 40 to between the body-facing layer 32 and the body portion 16 of the user. Prior to filling the fluid passage 40 with the heat transfer fluid 22 in one suitable embodiment, the body-facing layer 32 and outer layer 34 of the fluid passage lie flat against each other. Once the heat transfer fluid 22 flows inside the fluid passage 40, the cross-sectional area of the fluid passage increases to allow the heat transfer fluid to flow between the layers 32, 34. The layers 32, 34, in one embodiment, may be sealed to each other using heat sealing to provide adequate strength without requiring additional raw materials. Other methods of sealing the layers 32, 34 to each other, such as adhesive bonding, are also contemplated as being within the scope of the disclosure.

The laminate structure illustrated in FIG. 4 includes a porous layer 36, such as a layer of fibrous batting material. The fluid impermeable outer layer 34 retains the heat transfer fluid 22 within the garment 12, while the porous layer 36, which is a substantially hydrophobic material, allows the heat transfer fluid 22 to pass into direct contact with the body portion 16 for flow across the skin throughout the garment. In one embodiment, the porous layer 36 is bonded to the inner body-facing layer 32 such that the porous layer can be donned and removed with the other layers 32, 34 of the illustrated garment 12. In another embodiment, the porous layer 36 is separate from the other layers 32, 34 such that the porous layer can be donned and removed separately from the other layers 32, 34 of the illustrated garment 12.

In one suitable embodiment, a mesh body-facing layer 38 may be used to hold the porous layer 36 in place, allowing substantial contact between the body portion 16 and the heat transfer fluid 22 within the porous layer 36. In one embodiment, the fluid impermeable outer layer 34 includes a neoprene outer shell with an inner layer of aluminum-laminated polyester. The neoprene outer shell repels fluid, while the aluminum-laminated polyester facilitates insulating the garment 12. In another embodiment, the outer layer 34 is a transparent material such as polyvinyl chloride (PVC), polyethylene, or polyurethane that permits the body portion 16 to be seen through the garment 12. However, the outer layer 34 may include any material that enables the outer layer 34 to function as described herein. The porous layer 36 may include a polyester batting material, and the mesh body-facing layer 38 may include a nylon screen material. However, the porous layer 36 and the mesh body-facing layer 38 may include any material that enables the porous layer 36 and the mesh body-facing layer 38 to function as described herein.

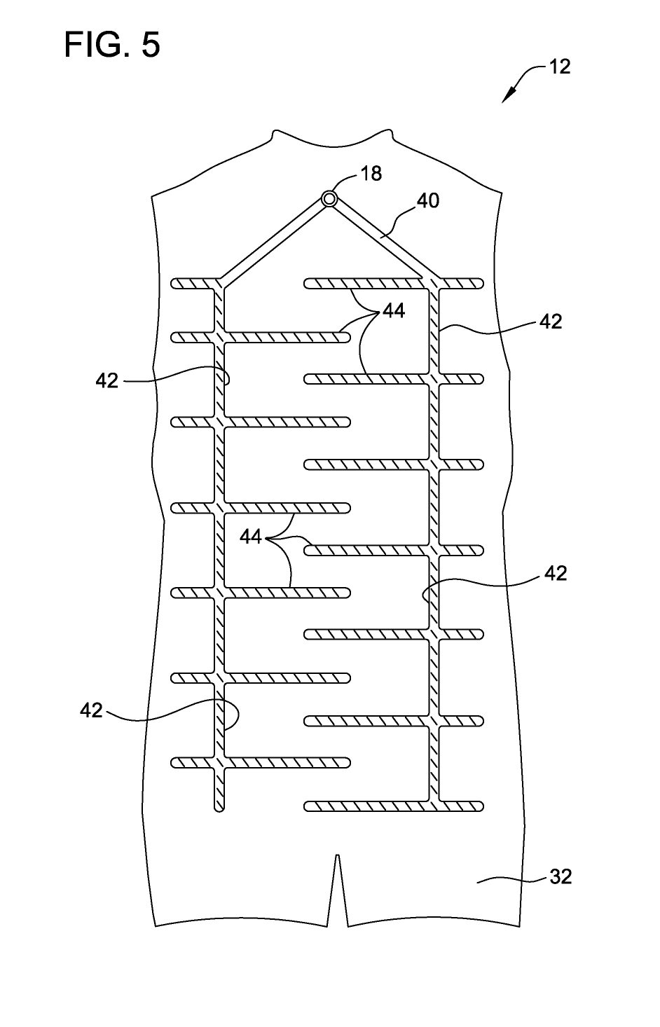

FIG. 5 is a schematic view of one embodiment of the body-facing layer of the garment having fluid passages formed therein. In the exemplary embodiment, the fluid passage 40 may be configured to distribute the heat transfer fluid 22 over substantially all of the body portion 16 of the user enclosed by garment 12. For example, the fluid passage 40 may include at least one main fluid passage 42 extending longitudinally of the garment 12, and at least one secondary fluid passage 44 extending laterally out from the main fluid passage 42. The main fluid passage 42 may branch into many secondary fluid passages 44 to further distribute the heat transfer fluid 22 to the body portion 16 of the user enclosed within the garment 12. The path of these passages 42, 44 may vary without departing from the scope of this disclosure.

With reference again to FIG. 4, in one embodiment, the layers 32, 34 may be further joined along their facing surfaces to form gas pockets 46. Gas pockets 46 are at least partially filled with a gas 48 (e.g., air) such that the pockets act as cushions to engage the body portion 16, holding an adjacent portion of the body-facing layer 32 slightly away from the body portion 16 of the user. The gas pockets 46 hold the body-facing layer 32 away from the body portion 16 of the user to facilitate the heat transfer fluid 22 movement between the body-facing layer 32 and the body portion 16 of the user. The gas pockets 46 may be rounded resulting in a limited contact area with the body portion 16 of the user so that the heat transfer fluid 22 may contact the user's skin, thus increasing the heat transfer effect of the heat transfer fluid 22. In other embodiments, fluid passages 40 extend abundantly throughout the garment 12, thus gas pockets 46 may not be necessary.

Referring to FIG. 2, the front panel 26 and the back panel 28, when joined at the plurality of seams (not shown), cooperate to form a neck opening 50 and limb openings 52 (e.g., openings for a user's arms and legs) in the garment 12. In the exemplary embodiment, the openings 50, 52 are configured for sealing engagement of the garment 12 with the body portions of the user extending therethrough. In one embodiment, the openings 50, 52 may include an elastic binding portion configured for sealing against the user's body and creating a fluid impervious seal. In other embodiments, the openings 50, 52 may be configured to sealingly engage a user's body portions extending therethrough using any method that enables the openings 50, 52 to function as described herein.

In one suitable embodiment, each of the limb openings 52 includes a built-in tourniquet 54. The tourniquets 54 can be selectively used to stop bleeding from the extremities (i.e., the arms and legs) of the user. The tourniquets 54 may also facilitate sealing the limb openings 52 of the garment 12 while the cooling system 10 is in use. Furthermore, the tourniquets 54 are adapted to reduce the flow of blood to and from the torso of the user and the extremities the user, which enhances the rate of cooling of the user.

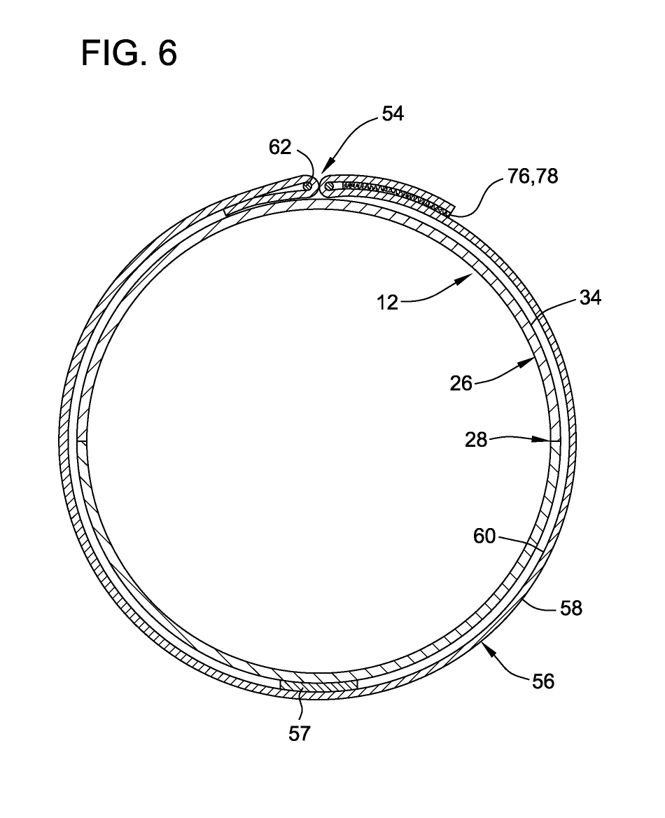

FIG. 6 is a cross-sectional view of an exemplary built-in tourniquet 54 for the limb openings 52 of the garment 12. In the exemplary embodiment, as illustrated, the garment 12 includes an outer layer 34. The tourniquet band 56, with outer surface 58 and inner surface 60 is located such that the outer layer 34 of the garment 12 is adjacent to but in no way obstructs the movement of the tourniquet band 56 such that when the tourniquet 54 is tightened or loosened, it is free to move. The tourniquet 54 has a fastening mechanism 62 attached to the tourniquet band 56 and located adjacent front panel 26 of garment 12. A portion of the tourniquet band 56 is attached to the back panel 28 of the garment 12 by, for example, sewing, adhesive bonding, heat welding, hook-and-loop fastening, or other attaching technique 57 to facilitate holding the tourniquet 54 to the garment when the tourniquet is unfastened or loosely fastened. In addition, the tourniquet 54 is configured to be manually tightened by the user by positioning the fastening mechanism 62 adjacent the front panel 26 and attaching a portion of the tourniquet band 56 to the back panel 28 of the garment 12. The fastening mechanism 62 permits the tourniquet band 56 circumference to be decreased in diameter to generate radial pressure on the limb of the user, i.e., an arm or a leg, to substantially reduce blood flow to the limb. The tourniquet 54, in one embodiment, is configured to exert a pressure equal to or greater than 180 millimeters of mercury (mmHg), i.e., a pressure equal to or greater than the arterial blood pressure of the user in a hypertensive state.

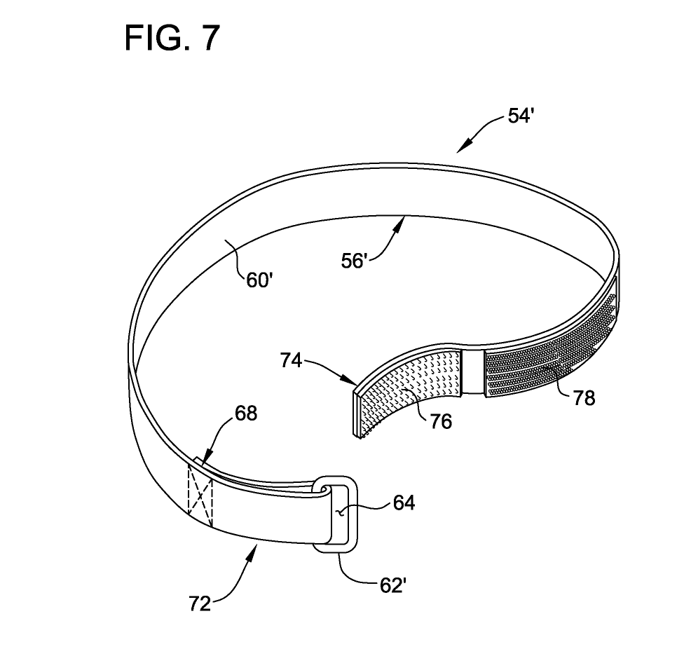

FIG. 7 is a perspective view of another exemplary tourniquet 54′ suited for use with the garment 12. In this embodiment, a fastening mechanism 62′ is configured as a loop where the opening 64 is configured to permit the tourniquet band 56′ to pass through unimpeded. The fastening mechanism 62′ may be made from metal, carbon fiber, or any similar material designed to withstand the forces necessary to enable fastening mechanism 62′ to function as described herein. The tourniquet band 56′ may be made from nylon or a similar material designed to withstand the forces necessary to enable tourniquet band 56′ to function as described herein. The tourniquet band 56′ is connected to the fastening mechanism 62′ by passing the inner surface 60′ of an end portion 68 of the tourniquet band 56′ through the fastening mechanism 62′. The end portion 68 is looped through opening 64 such that the inner surface 60′ of the end portion 68 is in face-to-face contact with a portion of the tourniquet band 56′. The portions where the tourniquet band 56′ overlap and are in face-to-face contact are attached to each other in any suitable manner, e.g., sewing, adhesive bonding, heat welding, or other suitable attaching technique, thus forming a loop 72 retaining the fastening mechanism 62′. In one embodiment, proximate an end portion 74, opposite end portion 68, of the tourniquet band 56′ is a temporary fastening means, e.g., a hook-and-loop fastening material 76, 78, for securing the tourniquet 54′ when in use. In operation, the end portion 74 is passed through the opening 64 of the fastening mechanism 62′, sufficient force to reduce substantially all of the blood flow to the limb is applied, and the end portion 74 is temporarily fastened to the tourniquet band 56′.

With reference again to FIG. 1, in one suitable embodiment, the cooling system 10 includes a portable control unit indicated at 20 for controlling operation of the cooling system 10. The portable control unit 20 can be compact, lightweight, integrated, and self-contained such that it can be worn by the user of the garment 12, or carried by one person to the user of the garment. The portable control unit 20 includes a liquid delivery system 80, a user interface 82, a power source 84, a fluid control system 86, and a valve system 88 for powering and controlling the cooling system 10. The portable control unit 20 may be useful where the cooling system 10 is to be used in the field or at a remote site where electricity is unavailable. In one embodiment, the power source 84 is a battery. In other embodiments, portable power sources, such as engine-based generators and motorized vehicles (e.g., electrical power derived from either) are also contemplated as potential sources of power. The portable control unit 20 is selectively attachable to the garment 12 such that a user may wear garment 12 without attachment of the portable control unit 20.

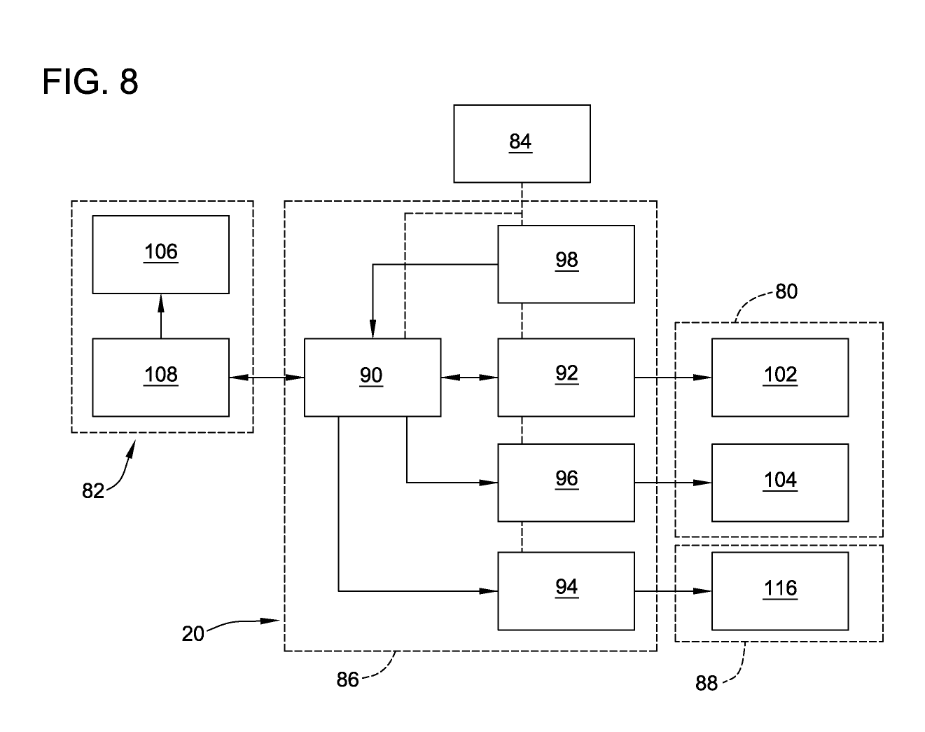

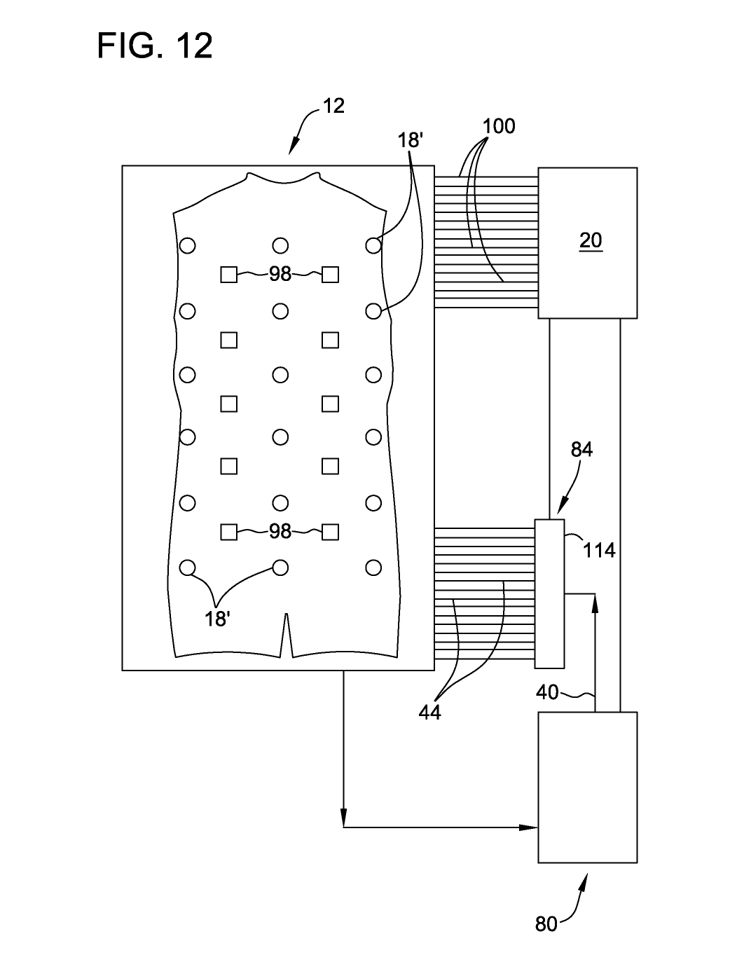

FIG. 8 is a schematic of the portable control unit 20 of the cooling system 10 of FIG. 1. The portable control unit 20 includes the fluid control system 86, having a programmable controller 90, an H-bridge drive circuit 92, a voltage limiter 94, at least one pump driver 96, and at least one sensor 98. The fluid control system 86 communicates with the liquid delivery system 80 and the valve system 88 to control the flow of the heat transfer fluid 22 through the garment 12. The sensors 98 (e.g., which may measure temperature, flow rate, or pressure) are positioned in the garment 12 for sensing a body temperature of the user and a property of the heat transfer fluid 22 within the garment 12. The portable control unit 20, which is integrated with the sensors 98 via control wires 100 (see FIG. 12), processes the body temperature reading of the user and the property of the heat transfer fluid 22 sensed by the sensors and correspondingly actuates the liquid delivery system 80 and the valve system 88 to distribute the heat transfer fluid 22 within the garment 12 in response to the sensed properties of the user and the heat transfer fluid 22. The fluid control system 86 is operable to alter the temperature of the heat transfer fluid 22 via a heat exchanger 102, drive a pump 104, and control the valve system 88. The cooling system further includes the user interface 82 for communicating the status of the system to the user, a display 106 for visually indicating particular parameters of the system, and controls 108 that allow the user of the system to control selectively particular system functions. For example, controls 108 may allow a set-point, or target, body temperature to be set. The display 106, for example, could display the set-point temperature along with the user's actual body temperature, the heat transfer fluid temperature, and the heat transfer fluid flow rate, among other things.

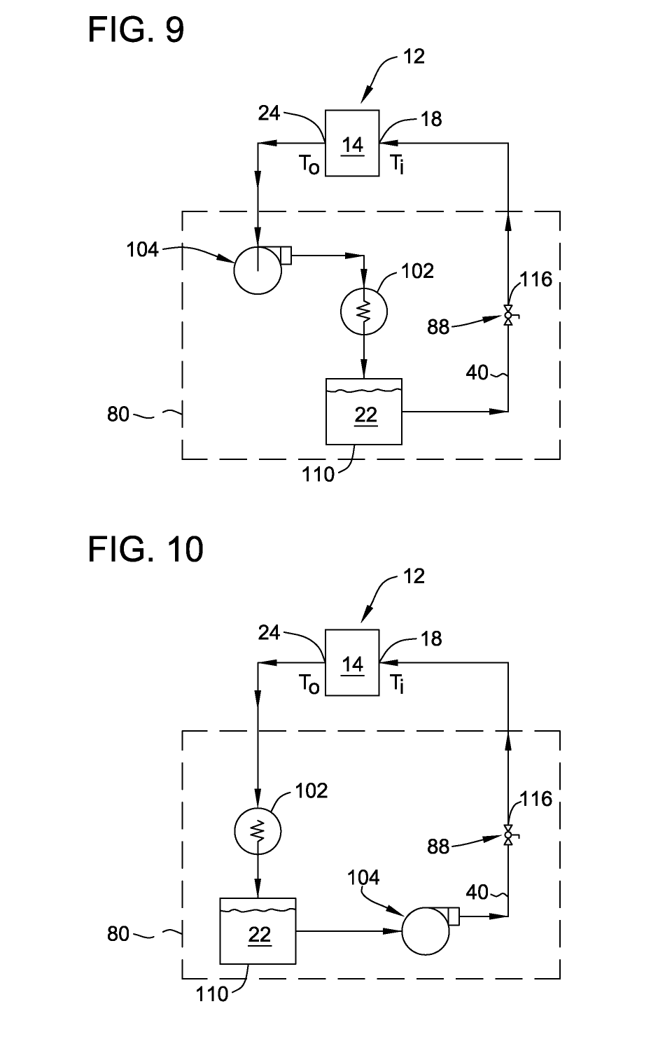

FIG. 9 is a schematic of one embodiment of the cooling system 10 including an outlet pump configuration. The liquid delivery system 80 channels the heat transfer fluid 22 through the inlet 18 of the garment 12, into the interior space 14, and through the outlet 24 of the garment 12. The liquid delivery system 80 includes the pump 104, the heat exchanger 102, a reservoir 110, and at least one fluid passage 40. The liquid delivery system 80 is a generally closed, continuous flow system in which heat transfer fluid 22 is cycled through interior space 14 of the garment via the outlet 24 and the inlet 18 of the garment 12. The pump 104 is connected in fluid communication with the outlet 24 for draining the heat transfer fluid 22 from the garment 12 and in fluid communication with the inlet 18 for channeling the heat transfer fluid 22 into the garment. In one embodiment, the pump 104 may be 12-volt direct current pumps having a pumping capacity of 2.4 liters per minute (0.63 gallons per minute). In another embodiment, the pumping capacity of such pumps may be increased to 3.0 liters per minute (0.79 gallons per minute) with 18 volts. In other embodiments, if different flow rates or other pump parameters are required, alternative pumps, such as gear, diaphragm, or centrifugal pumps, may be used without departing from the scope of the present invention. In one embodiment, the pump 104 is configured in an outlet pump arrangement drawing the heat transfer fluid 22 directly from the garment 12 and channeling it through the fluid passage 40 to the heat exchanger 102 and the reservoir 110 before channeling it through the inlet 18 and into the interior space 14 of the garment.

FIG. 10 is a schematic of another embodiment of the cooling system 10 including an inlet pump configuration. In this embodiment, the liquid delivery system 80 includes the pump 104 configured in an inlet pump arrangement to drive the heat transfer fluid 22 from the reservoir 110 directly into the inlet 18 of the garment 12 and to pull the heat transfer fluid 22 from the outlet 24 of the garment through the heat exchanger 102 and the reservoir 110. In another embodiment, the heat transfer fluid 22 may drain from the garment 12 directly to the reservoir 110 by gravity. From the reservoir 110, the heat transfer fluid 22 may be driven by the pump 104 through the garment inlet 18 back into the garment. In another embodiment, the liquid delivery system 80 may include both an outlet pump in fluid communication with the outlet 24 for draining the heat transfer fluid 22 from the garment 12 and an inlet pump in fluid communication with the inlet 18 for pumping the heat transfer fluid 22 into the garment 12. The heat exchanger 102 may be connected in fluid communication between the outlet pump and the inlet pump, such that heat transfer fluid 22 drained from the garment 12 by the outlet pump passes through the heat exchanger before entering the inlet pump.

The heat exchanger 102 of the liquid delivery system 80 is used to alter the temperature of the heat transfer fluid 22 to an inlet temperature Tᵢ, measured before the heat transfer fluid enters the garment 12. The heat transfer fluid 22 drained from the garment 12 may be reintroduced into the garment as described above after passing through the heat exchanger 102. The heat exchanger 102 alters the temperature of the heat transfer fluid 22 from an outlet temperature Tₒ measured after the heat transfer fluid exits the outlet 24 of the garment 12, to the inlet temperature Tᵢ. This allows the same heat transfer fluid 22 to be used repeatedly between the garment 12 and the liquid delivery system 80. Alternatively, the heat exchanger 102 may be positioned within the garment (not shown). In one embodiment, the heat transfer fluid 22 fluid is circulated through the heat exchanger 102 via a plurality of cooling channels formed within the heat exchanger. However, the heat exchanger 102 may use any configuration that enables the heat exchanger 102 to function as described herein, including, but not limited to, offset strip fin or pin fin configurations. In another embodiment, the heat exchanger 102 may be a thermoelectric cooling device, e.g., a Peltier device, used to remove or add heat energy to the heat transfer fluid 22. In another embodiment, heat exchanger 102 may incorporate a phase-change material (e.g., ice) to facilitate returning the heat transfer fluid 22 to its inlet temperature Tᵢ. In another suitable embodiment, heat exchanger 102 may incorporate hot or cold chemical packs to remove or add heat energy to the heat transfer fluid 22. It is understood that the heat exchanger 102 may be used to warm or cool the heat transfer fluid 22.

In an exemplary embodiment, as described above, the liquid delivery system 80 may include the reservoir 110 connected in fluid communication with the pump 104 and the heat exchanger 102, such that the heat transfer fluid 22 passes through the heat exchanger before it flows into the reservoir 110. In other embodiments, the relative positions of the reservoir 110 and the heat exchanger 102 may be reversed, such that the heat transfer fluid 22 from the garment 12 flows directly into the reservoir 110 for storage, until passing from the reservoir 110 and through the heat exchanger 102 immediately before reentering the garment 12. Such an arrangement may be useful if rapid changes in the heat transfer fluid 22 temperature were required.

In an exemplary embodiment, the reservoir 110 collects the heat transfer fluid 22 at the temperature induced by the heat exchanger 102 and stores it before the heat transfer fluid 22 is driven into the garment 12. In one embodiment, the reservoir 110 may be insulated to facilitate maintaining or altering the temperature of the heat transfer fluid 22 before it is driven into the garment 12. In one embodiment, the reservoir 110 has a capacity of about 4 liters (1.06 gallons). In other embodiments, the reservoir 110 may have a capacity of about 2 liters (0.53 gallons), or a capacity of about 1 liter (0.26 gallons), to ensure continued cycling of liquid through the cooling system 10. In one suitable embodiment, the reservoir 110 may be a canteen carried by the user. In other embodiments, reservoir 110 may be integrated with the liquid delivery system 80.

In one embodiment, referring back to FIG. 1, the reservoir 110 may include a temperature-altering component 112 in thermal communication with the heat transfer fluid 22 for altering the temperature of the heat transfer fluid 22. The temperature-altering component 112 may provide temperature stabilization once the heat transfer fluid 22 within the reservoir 110 reaches a specific temperature. The temperature-altering component 112 may be any material capable of absorbing or releasing heat, such as a phase-change material (e.g., ice) or hot/cold chemical packs. In one embodiment, the temperature-altering component 112 is in direct contact with the heat transfer fluid 22 within the reservoir 110. In another embodiment, the temperature-altering component 112 may be located in a separate compartment of the reservoir 110 and not in direct contact with the heat transfer fluid 22 within the reservoir 110.

In one embodiment, a thermoelectric cooling device, e.g., a Peltier device may be used to maintain separate compartments of the reservoir 110 at different temperatures. For example, a compartment configured for holding the temperature-altering component 112 may be maintained at −10° C. (14° F.) and the compartment configured for holding the heat transfer fluid 22 may be maintained at 2° C. (35.6° F.). The use of a thermoelectric cooling device inhibits the premature degradation of the temperature-altering component 112 while maintaining the heat transfer fluid 22 in a cooled state. As a result, the integrity of the temperature-altering component 112 is preserved and the heat transfer fluid 22 is maintained at a temperature suitable for direct skin contact with a user. Because at least a portion of the reservoir 110 directly contacts the heat transfer fluid 22, which has been in direct contact with the user, the reservoir 110, or a portion thereof, may be configured to be disposable to prevent cross-contamination to subsequent users.

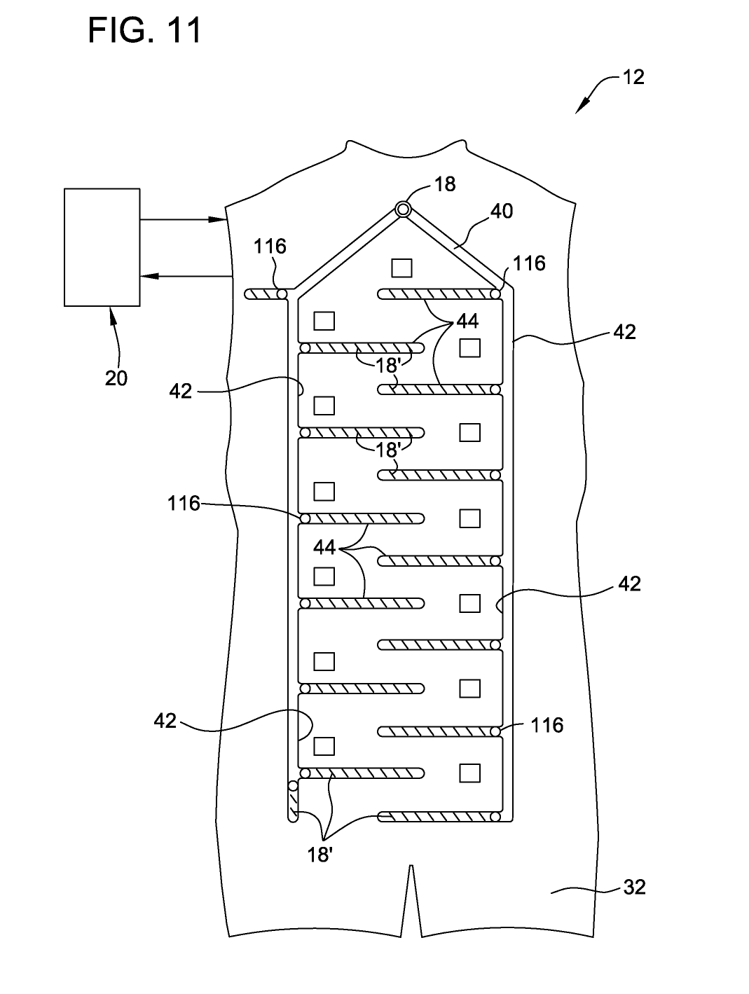

As described above, in one embodiment, the liquid delivery system 80 includes at least one fluid passage 40 selectively attachable for channeling the heat transfer fluid 22 to the inlet 18 and into the fluid passage 40 of garment 12. The fluid passage 40 is configured to distribute the heat transfer fluid 22 to fluid passage 40. FIG. 11 is a schematic of the portable control unit integrated with the valve control system and sensors for distributing the heat transfer fluid within the garment 12. The fluid passage 40 may include at least one main fluid passage 42 extending longitudinally of the garment 12, and at least two secondary passages 44 extending laterally out from the main passage 42. The main fluid passage 42 may branch into many secondary passages 44 connected with the sub-inlets 18′ to distribute liquid to the body portion 16 of the user within the garment 12. These passages may be insulated to inhibit heat transfer from the heat transfer fluid 22 flowing within the passages and the surrounding materials or ambient air.

FIG. 12 is a schematic of another embodiment of a portable control unit integrated with a valve control system and sensors for distributing the heat transfer fluid 22 within the garment 12. In this embodiment, the fluid passage 40 may enter a valve manifold 114 positioned outside the garment 12 which channels the heat transfer fluid 22 into a plurality of secondary passages 44. The secondary fluid passages 44 may be connected in fluid communication with sub-inlets 18′ to distribute liquid to the body portion 16 of the user within the garment 12. The paths of these passages may vary without departing from the scope of the disclosure.

Referring back to FIGS. 8 and 9, the liquid delivery system 80 includes the valve system 88 for controlling the flow of the heat transfer fluid 22 through the fluid passage 40. The valve system 88 includes at least one adjustable valve 116 operable to control the flow of the heat transfer fluid 22 within the liquid delivery system 80. The valve 116 is movable from a closed position that prevents the heat transfer fluid 22 from flowing, to an open position that permits the heat transfer fluid 22 to flow. In one embodiment, a valve 116 is associated with each sub-inlet 18′, thereby maximizing the potential flow patterns within the garment 12 (See FIG. 10). In another embodiment, the valve system 88 may be located remote from the sub-inlets 18′, as shown in FIG. 12 illustrating the valve manifold 114 located outside the garment 12.

In operation, the portable control unit 20 controls the liquid delivery system 80 and the valve system 88. The portable control unit 20 may regulate the flow rate of the heat transfer fluid 22 either by adjusting the pumping rate or by adjusting the valve 116. The heat transfer fluid 22 may be maintained at a flow rate between about 0.25 liters per minute (0.06 gallons per minute) and about 20 liters per minute (2.12 gallons per minute). In one embodiment, the heat transfer fluid 22 is controlled by the fluid control system 86 at a flow rate of about 1.5 liters per minute (0.40 gallons per minute).

The garment 12 is configured to allow the heat transfer fluid 22 to flow into the interior space 14 for direct contact with the user's body to promote heat transfer between the user and the heat transfer fluid 22. To raise the temperature of a user, the heat transfer fluid 22 is directed into the interior space 14 of the garment 12 at a temperature greater than the temperature of the body portion 16 of the user. For example, the heat transfer fluid 22 may have a temperature in a range of about 37° C. (99° F.) to about 47° C. (117° F.), such as about 45° C. (113° F.). One application of such a warming enclosure would be to warm a user suffering from unintended hypothermia.

To lower the temperature of a user, the heat transfer fluid 22 is directed into the interior space 14 of the garment 12 at a temperature lower than the temperature of the body portion 16 of the user received in the interior space 14 of the garment 12 so that the heat transfer fluid 22 cools the body portion 16 of the user. For example, the heat transfer fluid 22 may have a temperature in a range of about 0° C. (32° F.) to about 5° C. (41° F.), such as about 1° C. (34° F.). The heat transfer fluid 22 introduced into the garment 12 at such a temperature has been found to cool the body at a sufficient rate to induce hypothermia while minimizing any adverse effects to the skin of the user. It is to be understood that temperatures other than those listed above can be used to adjust the temperature of a user of the garment 12.

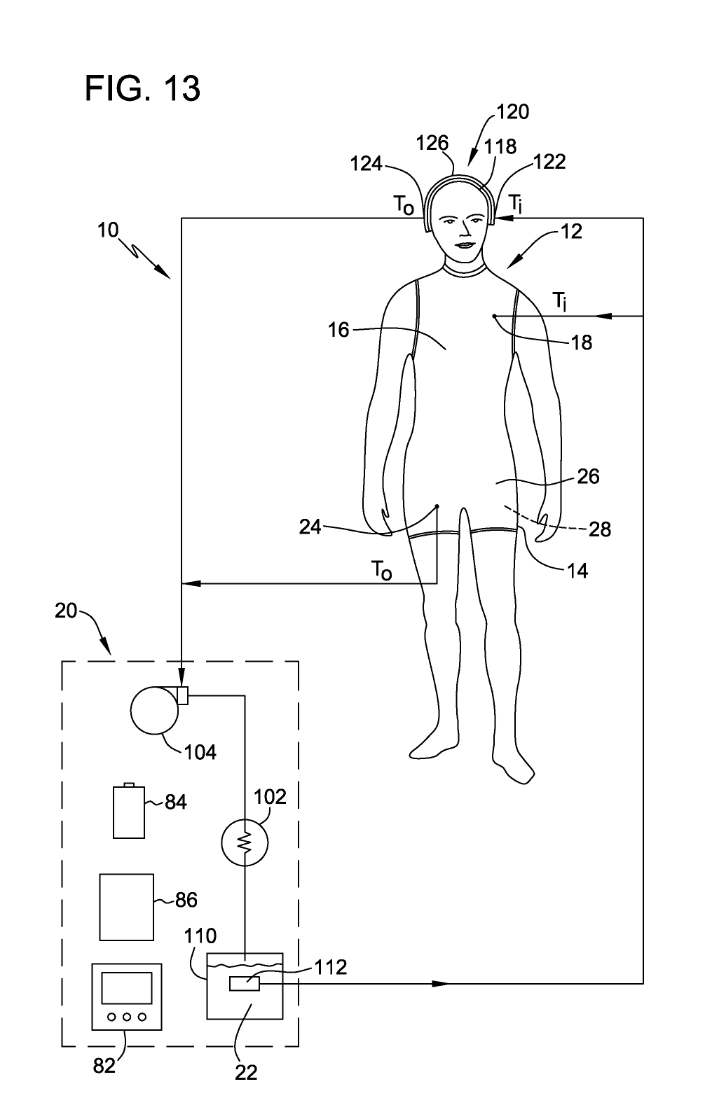

FIG. 13 is a schematic of one embodiment of the exemplary cooling system 10 including a head-cooling device. In one embodiment, the garment 12 may include a head-cooling device, generally indicated at 120, configured to engage the head of the user for circulating the heat transfer fluid 22 in direct contact with the head for extracranial cooling of the brain. The head-cooling device 120 may include a protective helmet 126 for placement upon the user's head. The protective helmet 126 is configured for sealing engaging the head of the user. The head-cooling device 120 includes a liner 118 within the helmet 126. The liner 118 is formed from substantially the same laminate structure used to form the garment 12. The head-cooling device 120 includes an inlet 122, providing a path for entry of the heat transfer fluid 22 for directly contacting the head, and an outlet 124, providing a path for draining the heat transfer fluid 22 from the head-cooling device 120. In one embodiment, the liner 118 is detachable from the inner surface of the helmet 126 for sanitary disposal of the liner.

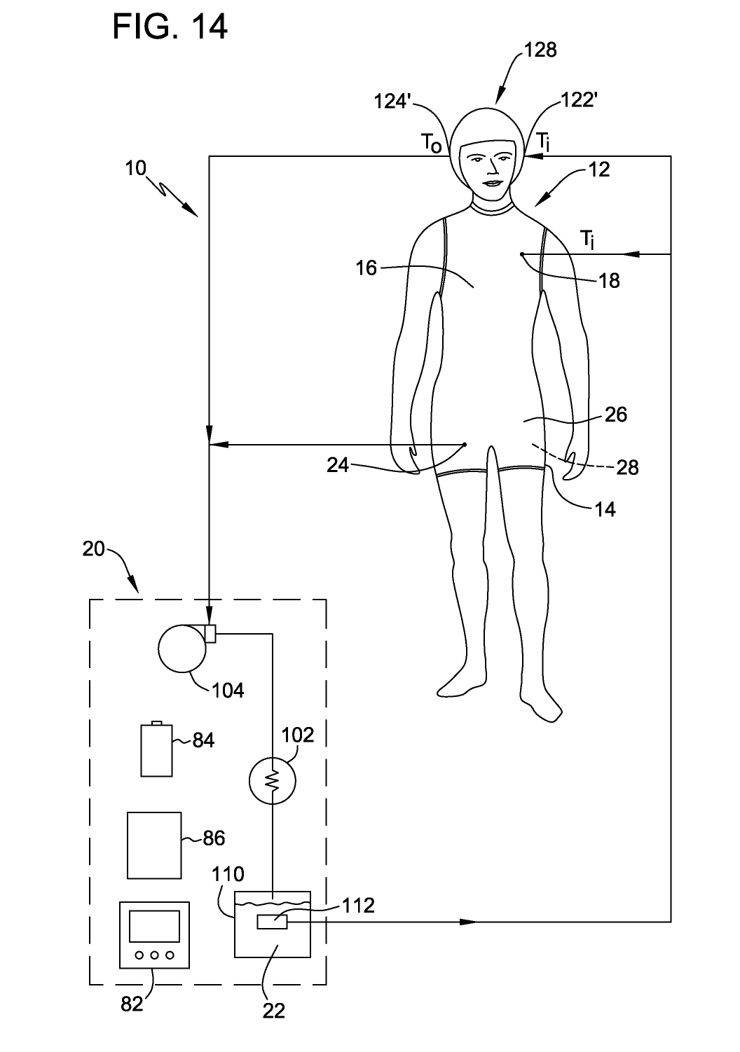

FIG. 14 is a schematic of another embodiment of the cooling system 10 including a head cooling device hood. The head cooling device hood 128 is attached to the garment 12 and is formed from substantially the same laminate structure used to form the garment 12. The head-cooling device hood 128 may include an inlet 122′, providing a path for entry of the heat transfer fluid 22 for directly contacting the head, and an outlet 124′, providing a path for draining the heat transfer fluid 22 from the head-cooling device hood 128. In another embodiment, the head cooling device hood 128 may be connected in fluid communication with garment 12. The head cooling device hood 128 sealingly engages the user's head for circulating the heat transfer fluid 22 in direct contact with the user's head.

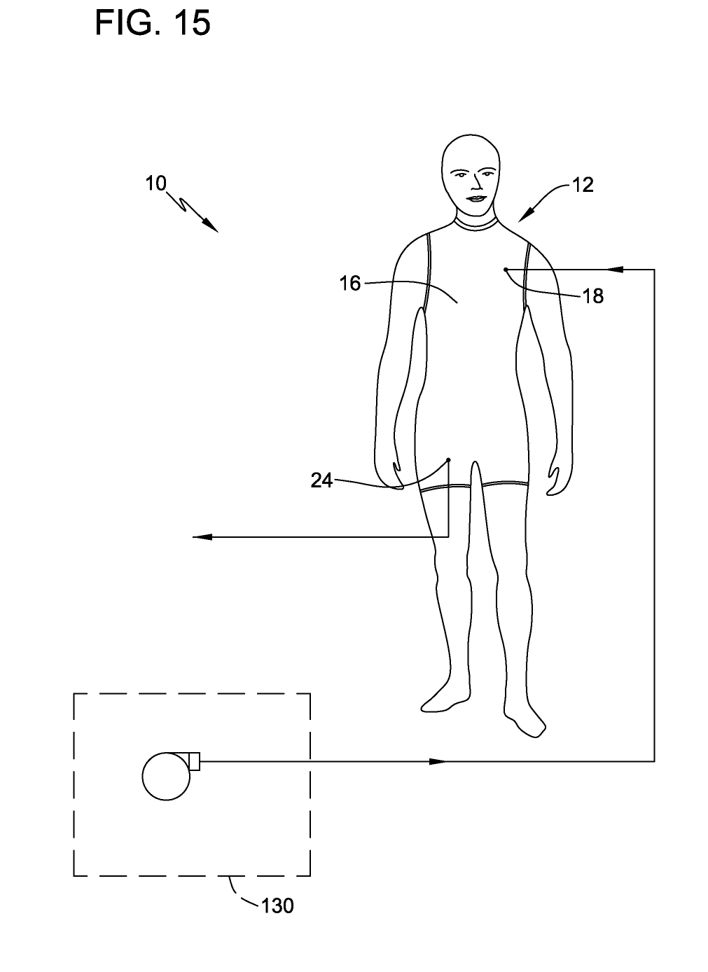

Additionally, in one embodiment, the cooling system 10 may include a portable blower apparatus 130 for circulating air through garment 12 to provide comfort to the user. FIG. 15 is a schematic of a blower apparatus 130 connected to the garment 12 of FIG. 2. The blower apparatus 130 is compact, lightweight, portable, and self-contained such that it can be worn by the user of the garment 12. It is contemplated that blower apparatus 130 is used for the user's comfort while wearing the garment 12 and not used with the portable control unit 20 during rapid cooling/warming of the user. The blower apparatus 130 is, preferably, battery powered and configured to supply ambient air to garment 12. The blower apparatus 130 may be useful where the garment 12 is worn by a user in the field or at a remote site where electricity is unavailable. The blower apparatus 130 is selectively attachable to the garment 12 such that a user may wear garment 12 with or without attachment of the blower apparatus 130. In one embodiment, the blower apparatus 130 drives ambient air through the inlet 18 of the garment 12, into the interior space 14, and through the outlet 24 of the garment 12 to the atmosphere. It is also understood that the blower apparatus 130 can include or be coupled to a portable air cooling or air heating device for cooling or heating the air prior to be driven into the garment 12. In such an embodiment, the garment 12 can be used to maintain the body temperature of the user at a comfortable temperature (e.g., in hot or cold environments). It is thus understood that the illustrated garment 12 can be selectively used with either the blower apparatus 130 or the portable control unit 20.

Each of the following U.S. patents and patent applications, which are assigned to Life Recovery Systems HD, LLC, describes apparatus and methods suitable for altering the body temperature and include U.S. Patent Application Publication No. 2008-0221493; U.S. Patent Application Publication No. 2011-0208275; U.S. Patent Application Publication No. 20110238143; U.S. Pat. No. 5,755,756; U.S. Pat. No. 6,969,399; U.S. Pat. No. 7,377,935; U.S. Pat. No. 7,547,320; U.S. Pat. No. 7,666,213; U.S. Pat. No. 7,731,739; U.S. Pat. No. 7,771,461; U.S. Pat. No. 7,892,271; and U.S. Pat. No. 8,182,520. Each of the above-listed U.S. patents and patent applications is incorporated herein by reference in their entireties.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

What is claimed is:

1. A system for maintaining and altering the body temperature of a user, the system comprising:
a garment defining an interior space adapted to conform to and enclose a torso of the user, the garment comprising at least one fluid passage configured to channel a heat transfer fluid into direct contact with the torso of the user;
a portable pump for circulating a heat transfer liquid through the at least one fluid passage of the garment; and
a portable blower apparatus for circulating a heat transfer gas through the at least one fluid passage of the garment.

2. The system in accordance with claim 1, further comprising at least one inlet for receiving the heat transfer liquid and the heat transfer gas into the garment and at least one outlet for allowing the heat transfer liquid and the heat transfer gas to exit the garment.

3. The system in accordance with claim 1, wherein the garment further comprises a head cooling device coupled in fluid communication with the portable pump, the head cooling device being configured to channel the heat transfer liquid and the heat transfer gas into direct contact with a head of the user.

4. The system in accordance with claim 3, wherein the head cooling device is liner adapted to be worn under a protective helmet.

5. The system in accordance with claim 3, wherein the head cooling device is separate from the garment.

6. The system in accordance with claim 1, wherein the garment further comprises a porous layer engageable with the torso of the user for carrying at least one of the heat transfer liquid and the heat transfer gas throughout the garment, the porous layer being formed from a substantially hydrophobic material.

7. The garment in accordance with claim 1, wherein the heat transfer liquid has a volume between about 1 liters and about 2 liters.

8. A system for maintaining and altering the body temperature of a user, the system comprising:
a garment defining an interior space adapted to conform to and enclose a torso of the user and having a pair of arm openings and a pair of leg openings, the garment comprising at least one fluid passage configured to channel a heat transfer fluid into direct contact with the torso of the user;
a portable pump for circulating the heat transfer fluid through at least one fluid passage of the garment; and
a tourniquet positioned at each of the leg and arm openings, each of the tourniquets being selectively moveable between an inoperable and an operable position wherein the tourniquet is capable of reducing blood flow to the respective arm or leg of the user.

9. The system in accordance with claim 8, wherein each of the tourniquets is configured to be manually tightened and is operable independent of the other tourniquets.

10. The system in accordance with claim 8, wherein each of the tourniquets is adapted to be manually tightened to exert a pressure equal to or greater than 180 millimeters of mercury (mmHg) to the respective arm or leg.

11. The system in accordance with claim 8 wherein each of the tourniquets is built into the garment.

12. The system in accordance with claim 8, wherein the garment further comprises a porous layer formed from a substantially hydrophobic material, the porous layer being engageable with the torso of the user to provide a porous surface adjacent the torso.

13. The system in accordance with claim 8, further comprising body armor garment, the garment being configured to be worn under the body armor garment.

14. The system in accordance with claim 13, further comprising a helmet.

15. A system for maintaining and altering the body temperature of a user, the system comprising:
an outer body armor garment configured to protect at least a torso of the user;
a garment for placement under the outer body armor garment, the garment defining an interior space adapted to conform to and enclose the torso of the user, the garment comprising at least one fluid passage configured to channel a heat transfer fluid into direct contact with the torso;
a helmet configured for configured to protect the head of the user, the helmet comprising a liner having at least one fluid passage configured to channel the heat transfer fluid into direct contact with the head of the user; and
a portable pump for circulating the heat transfer fluid through the at least one fluid passage of the garment and the at least one fluid passage of the liner.

16. The system in accordance with claim 15, wherein the liner is detachable from the helmet.

17. The system in accordance with claim 15, wherein the garment comprises an outer layer formed from a liquid impermeable material and a porous layer formed from a substantially hydrophobic material, the porous layer being engageable with the torso of the user to provide a porous surface adjacent the torso.

18. The system in accordance with claim 17, wherein the porous layer formed is separate from the outer layer.

19. The system in accordance with claim 15, further comprising a liquid reservoir in fluid communication with the portable pump.

20. The system in accordance with claim 19, wherein the liquid reservoir is a canteen.
